(19) United States
(12) Patent Application Publication
(10) Pub. No.: US 2015/0376040 A1
(43) Pub. Date: Dec. 31, 2015

(54) ADDITIVES FOR OLEFIN PLANT QUENCHING FLUID pH CONTROL

(71) Applicant: BAKER HUGHES INCORPORATED, Houston, TX (US)

(72) Inventor: Roger D. Metzler, Sugar Land, TX (US)

(73) Assignee: BAKER HUGHES INCORPORATED, Houston, TX (US)

(21) Appl. No.: 14/316,279

(22) Filed: Jun. 26, 2014

(51) Int. Cl. C02F 1/66; C07C 7/148

(52) U.S. Cl. CPC C02F 1/66; C07C 7/14875; C07C 7/1485; C02F 2209/06

(57) ABSTRACT

A neutralizing additive composition may contact a fluid, such as a quench medium composition, an effluent stream, a dilution steam condensate, a dilution steam, and combinations thereof for neutralizing any neutralizable components therein. A non-limiting example of neutralizing components may be organic acids. The neutralizing additive may be or include at least one strong base and at least one amine. The strong bases(s) may be or include metal hydroxides, metal carbonates, and combinations thereof.

### TECHNICAL FIELD

The present invention relates to a neutralizing additive composition for use in a fluid, such as a quench medium, effluent stream, dilution steam condensate, dilution steam, and the like; more specifically, the invention relates to a neutralizing additive having at at least one strong base and at least one amine where the strong base may be or include metal hydroxides, metal carbonates, and combinations thereof.

### BACKGROUND

Converting oxygenates into light olefins is referred to as the oxygenate-to-olefin process. Non-limiting examples of oxygenates include methanol, ethanol, n-propanol, isopropanol, methyl ethyl ether, dimethyl ether, diethyl ether, di-isopropyl ether, formaldehyde, dimethyl carbonate, dimethyl ketone, acetic acid, and mixtures thereof. Non-limiting examples of olefins may be or include ethylene, propylene, and the like. Olefins are important petrochemicals used to make plastics or other chemicals, such as vinyl chloride, ethylene oxide, ethylbenzene, alcohol, acrylonitrile, propylene oxide, and the like.

There are numerous technologies available for producing oxygenate(s), including fermentation or reaction of synthesis gas derived from natural gas, petroleum liquids, carbonaceous materials including coal, recycled plastics, municipal waste, or any other organic material. To produce methanol, a combustion reaction of natural gas, mostly methane, and an oxygen source having hydrogen, carbon monoxide and/or carbon dioxide produces synthesis gas. Synthesis gas production processes are well known, and include conventional steam reforming, autothermal reforming or a combination thereof. The synthesis gas may then be converted into methanol, for use in the oxygenate-to-olefin process.

The oxygenate-to-olefins reaction is highly exothermic and may have a large amount of water. The effluent stream derived from an oxygenate-to-olefin process may include as much as one half of the total weight of the effluent stream as water. Consequently, the water must be removed by condensation in a quench device or a quench tower to isolate the olefin product. The stream is considered an effluent stream from the point the effluent stream exits an oxygenate-to-olefin reactor to the point the gaseous output stream is quenched; quenching the effluent stream produces a quenched effluent stream.

As used herein, “quench device”, also known as a “quench tower”, is a device for introducing a sufficient quantity of liquid quench medium to a gaseous effluent stream where the quench medium may condense at least a portion of the material in the effluent stream. A quench tower is a type of quench device having more than one quench stage. A “quench medium” is defined as a liquid that contacts the effluent stream to cool the effluent stream to the condensation temperature of water.

Generally, the step of quenching the effluent stream forms a quenched effluent stream and a liquid fraction or quench bottoms stream. As used herein, the terms, “liquid fraction” and “quench bottoms stream” are used interchangeably and refer to the portion of the effluent stream and quench medium that is liquid under quench conditions and includes all streams that contain the condensed portion of the effluent stream and fractions of the condensed effluent stream. The term “quenched effluent stream” refers to the portion of the effluent stream that is predominantly gaseous after at least one stage of quenching.

Water from the quench bottoms stream may be treated or processed to remove the entrained hydrocarbons (e.g. quench oil, pyrolysis, gasoline, etc.) and potential coke fines that may foul heat exchangers and boilers, lead to poor separation in stripping units, increase energy consumptions, and the like. After treatment to the water, the water may be fed into a dilution steam system where steam may be added to the quenched effluent stream to reduce the partial pressure of hydrogen and shift the equilibrium toward a higher olefin yield. Dilution steam condensate is defined herein as the water condensed from a quench device prior to being treated for use in a dilution steam system, which is different from a dilution steam, i.e. water condensed from the quench device that has been treated for use in a dilution steam system.

The quench medium, the effluent stream, dilution condensate, and/or the dilution steam often contain byproducts including oxygenate byproducts, such as carbon dioxide, organic acids, aldehydes, and/or ketones. Furthermore, depending upon operating conditions, unreacted oxygenates may be present in the effluent stream of the oxygenate-to-olefin reaction.

Neutralizing additives may contact the quench medium, the effluent stream, and/or dilution steam to alter the pH thereof. The use of neutralizing additives in such a fluid has been difficult to control due to varying objectives throughout the oxygenate-to-olefin process or system, such as pH control and/or volatility. Strong bases, such as sodium hydroxide and ammonia (both ammonium hydroxide and anhydrous ammonia) have been used, but these are difficult to control within pH target ranges. These strong bases often carry over into the dilution steam, which creates problems within the dilution steam system and crackers. Sodium carry over into a furnace is a major concern due to coking, carbon monoxide, and tubing cracking issues. Amines have been employed as an attempt to decrease the use of sodium-containing strong bases, but also have technical limits, and are more expensive than the sodium-containing strong bases.

It would be desirable if alternative neutralizing additives were devised to alleviate some of these problems.

### SUMMARY

There is provided, in one form, a neutralizing additive composition for use in a fluid, such as but not limited to a quench medium, an effluent stream, a dilution condensate, a dilution steam, and combinations thereof. The neutralizing additive may have or include at least one strong base and at least one amine. The strong base(s) may be or include, but are not limited to, metal hydroxides, metal carbonates, and combinations thereof.

There is further provided in another non-limiting embodiment a treated fluid composition having a fluid and a neutralizing additive. The fluid may have or include neutralizable components, and the fluid may be or include a quench medium, an effluent stream, a dilution condensate, a dilution steam, and combinations thereof. The neutralizing additive may be present in the treated fluid composition in an amount effective to neutralize at least a portion of the neutralizable components. The neutralizing additive may have or include at least one strong base and at least one amine. The strong base may be or include metal hydroxides, metal carbonates, and combinations thereof. More of the neutralizable components may be neutralized within the treated fluid composition as compared to an otherwise identical fluid composition absent the neutralizing additive composition.

In another embodiment, a method may include contacting a fluid having neutralizable components with a neutralizing additive composition in an effective amount to neutralize at least a portion of the neutralizable components. The method may also include neutralizing at least a portion of the neutralizable components within the fluid. The fluid may be or include a quench medium, an effluent stream, a dilution steam condensate, a dilution steam, and combinations thereof. The neutralizing additive may include at least one strong base and at least one amine. The strong base(s) may be or include metal hydroxides, metal carbonates, and combinations thereof.

The neutralizing additive may be better suited for the fluid, as well as decrease corrosion to the equipment used in an oxygenate-to-olefin system compared to other strong bases used for neutralizing such fluids.

### BRIEF DESCRIPTION OF THE DRAWINGS

FIG. 1 is a graph illustrating the correlation between the volume of monoethanolamine (MEA) added and the pH associated therewith;

FIG. 2 is a graph illustrating the correlation between the volume of different neutralizer additives and the pH associated with the volume of each additive;

FIG. 3 is a graph illustrating the correlation between the volume of different neutralizer additives and the pH associated with the volume of each additive;

FIG. 4 is a graph illustrating the correlation between the volume of different neutralizer additives and the pH associated with the volume of each additive;

FIG. 5 is a graph illustrating the correlation between the volume of different neutralizer additives and the pH associated with the volume of each additive;

FIG. 6 is a schematic representing a typical olefins plant quench water/dilution steam system; and

FIG. 7 is a schematic similar to FIG. 6, but representative pH targets are shown.

### DETAILED DESCRIPTION

It has been discovered that a neutralizing additive composition having a strong base and an amine, may contact a fluid to neutralize any neutralizable components present within the fluid. The contacting may occur by adding, circulating, and/or injecting the neutralizing additive into the fluid. The fluid may be or include, but is not limited to, a quench medium, an effluent stream, a dilution steam condensate, a dilution steam, and combinations thereof.

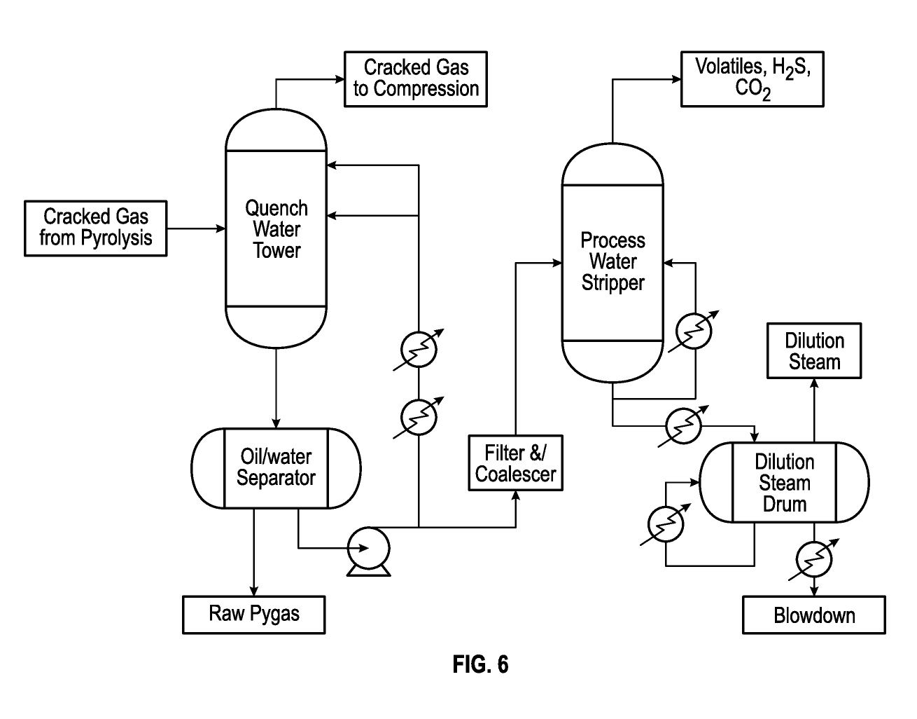

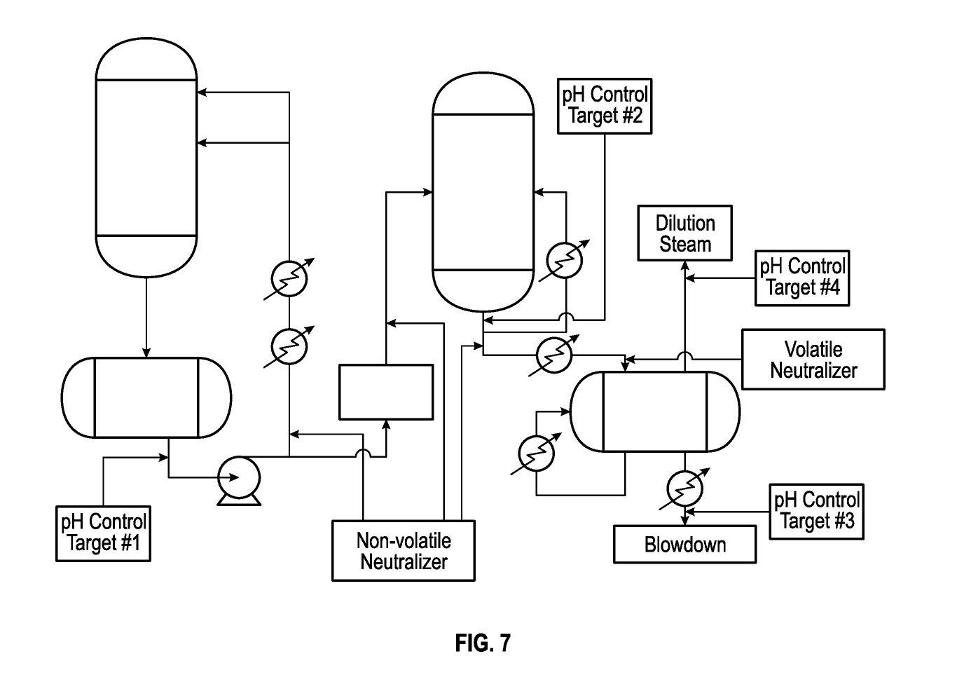

FIG. 6 is a non-limiting representation of a typical olefins plant quench water/dilution steam system. In a non-limiting embodiment, the neutralizing additive may be added to either the quench medium (within the quench water tower), the effluent stream flowing from the quench water tower, the effluent from the process water stripper, the effluent from the dilution steam drum, the effluent from the dilution steam, and combinations thereof as depicted in FIG. 7. Alternatively, the neutralizing additive may be added to the effluent stream prior to quenching the effluent stream to slightly adjust the pH of the effluent stream prior to contacting the effluent stream with the quench medium. Alternatively, the neutralizing additive may contact the effluent stream, dilution steam condensate, dilution steam, and combinations thereof after a removal of carbon dioxide from the quenched effluent stream has occurred to further neutralize or adjust the pH of the fluid.

The fluid may include water, hydrocarbons, neutralizable components, and combinations thereof. In a non-limiting embodiment, the quench medium may include quench oil or water. The fluid may be in a liquid state, a gaseous state, and combinations thereof.

It is not necessary for the neutralizable components to be entirely inactivated or neutralized for the methods, neutralizing additive compositions, and/or treated fluid compositions to be considered effective, although complete neutralization is a desirable goal. Success is obtained if more of the neutralizable components are neutralized by contacting the fluid with the neutralizing additive than in the absence of the neutralizing additive.

Alternatively, the methods described are considered successful if a majority of the neutralizable components within the fluid are neutralized. ‘Majority’ is defined herein to be an amount greater than about 50% of the neutralizable components. According to one embodiment, contacting the fluid with the neutralizing additive may neutralize about 90 wt % or more of the neutralizable components, alternatively from about 95 wt % or more, or from about 98 wt % or more in another non-limiting embodiment.

The amount of the components within the neutralizing additive may vary depending on the desired characteristics of the fluid, and the location of the fluid within the oxygenate-to-olefin system. In a non-limiting example, the pH of the fluid may range from about 5 independently to about 7 where more of the amine may be included in the neutralizing additive instead of the strong base to obtain and/or maintain this range for better oil/water separation. Alternatively, it may be desirable for the fluid to have a pH ranging from about 8.8 independently to about 9.5, and more of the strong base (e.g. potassium base in a non-limiting example) may be included in the neutralizing additive for buffering the pH of the dilution steam instead of the amine. The individual pH ranges may be difficult to obtain and/or maintain when using only the amine (in the absence of the strong base) or only the strong base (e.g. in the absence of the amine). As used herein with respect to a range, “independently” means that any threshold may be used together with another threshold to give a suitable alternative range, e.g. about 5 independently to about 8.8 is also considered a suitable alternative range for the pH range.

However, to give an idea of potential pH ranges for the fluid, the pH of the fluid as it enters the quench device or dilution steam system may range from about 5 independently to about 11.5, alternatively from about 7.1 independently to about 11, or from about 8 independently to about 10 in another non-limiting embodiment. In yet another non-limiting embodiment, the pH of the fluid may be about 9. The temperature of the quench medium may range from about 60° F. (15° C.) independently to about 200° F. (93° C.); alternatively from about 80° F. (27° C.) independently to about 140° F. (60° C.); or about 110° F. (43° C.) in another non-limiting embodiment.

In another non-limiting embodiment, the strong base may be added at the same time or a different time from the amine depending on the desired characteristics of the fluid (e.g. pH range, volatility, etc.), cost of the neutralizing additive, flow rate of the fluid, and the like. Alternatively, the strong base may be added at the same location or a different location from the amine depending on the aforementioned desired characteristics of the fluid.

It is very difficult to estimate the amount of neutralizing additive to be added to the fluid because the amount is dependent upon the total acid/base loading to the stream and desired pH for the fluid within a zone of the fluid stream. Within the neutralizing additive, the amount of a potassium hydroxide may range from about 1 wt % independently to about 60 wt %, alternatively from about 10 wt % independently to about 40 wt %. The amount of the amine within the neutralizing additive may range from about 1 wt % independently to about 80 wt %, alternatively from about 1 wt % independently to about 50 wt %. The ratio of the strong base to the at least one amine may range from about 1:60 independently to about 60:1, or from about 1:5 independently to about 5:1 in another non-limiting embodiment.

A strong base is defined herein to be a base that completely dissociates in water into its cation and anion components. Non-limiting examples of the strong bases usable in the neutralizing additive may be or include, but are not limited to, metal hydroxides, metal carbonates, and combinations thereof. The metal hydroxides may be or include, but are not limited to, sodium hydroxide, calcium hydroxide, potassium hydroxide, lithium hydroxide, magnesium hydroxide, and combinations thereof. The metal carbonates may be or include, but are not limited to, sodium carbonate, calcium carbonate, potassium carbonate, lithium carbonate, magnesium carbonate, and combinations thereof. In a non-limiting example, sodium-containing strong bases are not used within the neutralizing additive. Those skilled in the art of quench devices and dilution steam systems would understand when to use a sodium-containing strong base within the neutralizing additive. The amine(s) may be or include, but are not limited to, triethylenetetramine (TETA), tetraethylenepentamine (TEPA), diethanolamine (DEA), ethanolamine (MEA), methoxypropylamine (MOPA), morpholine, cyclohexylamine, and combinations thereof.

A non-limiting example of the neutralizable components may be or include organic acids, although any acid capable of being neutralized by a strong base and an amine are included within the definition of neutralizable components. Organic acids are defined herein as acids having one or more carbon atoms that have at least one C—H bond, such as but not limited to formic acid, acetic acid, propanoic acid, butyric acid, and mixtures thereof. Unless otherwise specifically mentioned, the term neutralizable component or organic acid, as well as specific types of neutralizable components or organic acids, include the salt form. In the neutral salt form, the acids may be more soluble in water and may be more likely to be extracted in the bottoms stream of a quench device.

Once the neutralizing additive contacts the quench medium in a non-limiting embodiment, the quench medium may contact the effluent stream to neutralize or ‘quench’ the effluent stream, i.e. cool the effluent stream to separate or condense water from the effluent stream and produce a quenched effluent stream. The quenched effluent stream may undergo a collective step of washing and drying to produce a dried effluent stream. Washing the quenched effluent stream may separate acidic components, such as carbon dioxide, with an alkaline wash. Drying the quenched stream may follow washing and may remove saturated water in the quenched effluent stream. Optionally, the washing and drying stage may include other processing steps to remove additional oxygenates. One or more olefin products may be removed from the dried effluent stream.

The volume of quench medium added to an effluent stream expressed as a weight percentage of the total amount of water in the effluent stream entering the first quench stage may range from about 10 wt % independently to about 30 wt %; or from about 20 wt % independently to about 30 wt %; or about 25 wt % in another non-limiting embodiment. In one embodiment, contacting the effluent stream with a quench medium may remove at least 95 wt % or more of the water, alternatively from about 98 wt % or more, or from about 99 wt % or more from the effluent stream in another non-limiting embodiment based upon the total amount of water in the effluent stream prior to contacting the effluent stream with the quench medium.

The quench device in one embodiment may have single or multiple stages. The number of stages may range from about one stage independently to about four stages, or from about two stages independently to about three stages in another non-limiting embodiment. The quench device may be or include a device having single or multiple housings or towers. The quench device may include internal elements in a contact zone to facilitate intimate contacting of the quench medium with the effluent stream or portions thereof in a non-limiting embodiment. Contact zone is defined as the zone of the quench device where the quench medium may contact the effluent stream. Internal elements of the quench device may include liquid distributors and contacting devices, such as baffles, trays, random packing, or structured packing.

The quench device bottoms temperature may range from about 180° F. (82° C.) independently to about 300° F. (149° C.); or from about 180° F. (82° C.) independently to about 250° F. (121° C.); alternatively about 200° F. (93° C.). The quench device may be operated at a pressure that is from about 15 psig (103 kpag) independently to about 50 psig (345 kPag); or from about 15 psig (103 kPag) independently to about 40 psig (276 kPag); or about 20 psig (138 kPag) in another non-limiting embodiment.

The water separated from the quench bottoms stream, i.e. a dilution steam condensate, may be further treated for use in a dilution steam system. The neutralizing additive may contact the dilution steam condensate and/or the dilution steam (i.e. the dilution steam condensate after treatment) to neutralize any neutralizable components therein that may cause potential problems in the dilution steam system.

The invention will be further described with respect to the following Examples which are not meant to limit the invention, but rather to further illustrate the various embodiments.

### Example 1

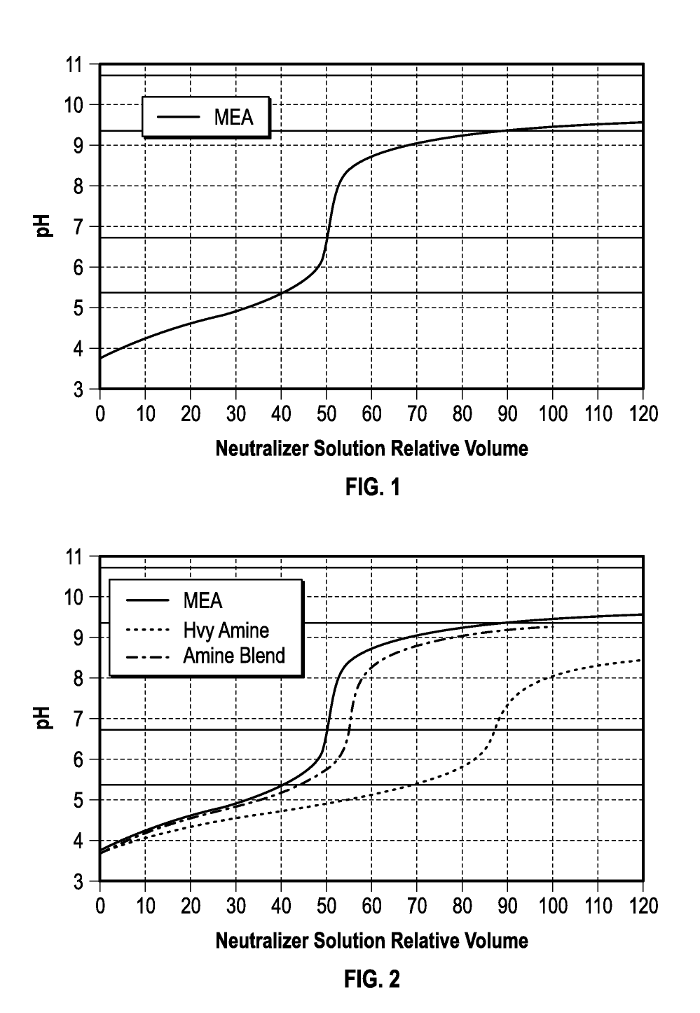

FIG. 1 is a graph illustrating the correlation between the volume of monoethanolamine (MEA) added and the pH associated therewith.

### Example 2

FIG. 2 is a graph illustrating the correlation between the volume of different neutralizer additives and the pH associated with the volume of each additive. The neutralizer additives were MEA, a heavy amine, and an amine blend. The heavy amine was diethanolamine (DEA). The amine blend was 80 wt % MEA and 20 wt % DEA.

### Example 3

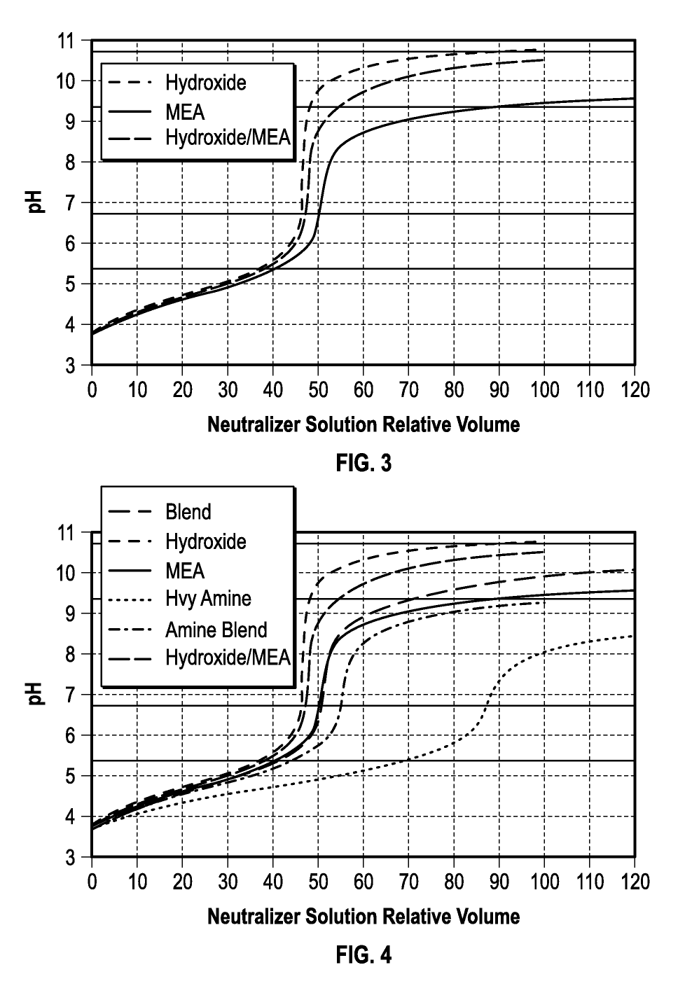

FIG. 3 is a graph illustrating the correlation between the volume of different neutralizer additives and the pH associated with the volume of each additive. The neutralizer additives were a hydroxide, MEA, and a blend of hydroxide and MEA. The hydroxide was potassium hydroxide (KOH). The hydroxide/MEA blend was 40 wt % hydroxide and 60 wt % MEA.

### Example 4

FIG. 4 is a graph illustrating the correlation between the volume of different neutralizer additives and the pH associated with the volume of each additive. The neutralizer additives were a hydroxide/MEA/DEA blend, a hydroxide, MEA, a heavy amine, an amine blend, and a blend of hydroxide and MEA. The hydroxide/MEA/DEA blend included 40 wt % potassium hydroxide (KOH), 50 wt % MEA, and 10 wt % DEA. The hydroxide was KOH. The heavy amine was DEA. The amine blend included 80 wt % MEA and 20 wt % DEA. The hydroxide/MEA blend was 40 wt % hydroxide and 60 wt % MEA.

### Example 5

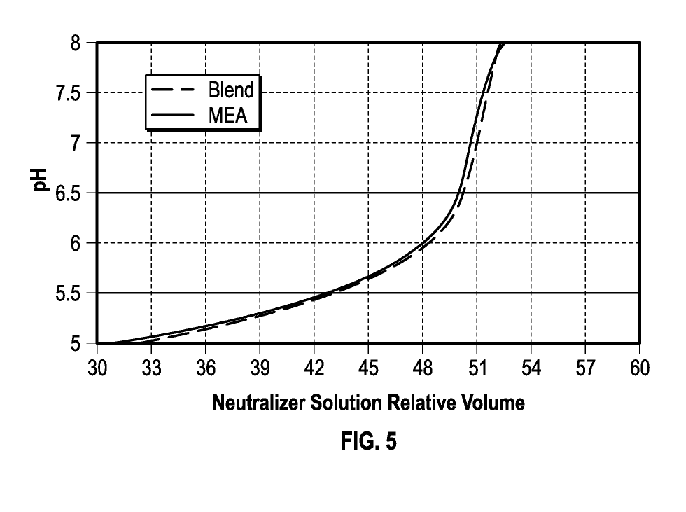

FIG. 5 is a graph illustrating the correlation between the volume of different neutralizer additives and the pH associated with the volume of each additive. The neutralizer additives were a hydroxide/MEA/DEA blend, and a MEA. The hydroxide/MEA/DEA blend included 40 wt % KOH, 50 wt % MEA, and 10 wt % DEA.

As noted from Examples 1-5, different neutralizing additives have different pH profiles. Thus, different neutralizing additives may be used to obtain a different pH ranges desired within a particular zone of the quench water/dilution steam system.

In the foregoing specification, the invention has been described with reference to specific embodiments thereof, and has been described as effective in providing additive compositions, treated fluid compositions, and methods for contacting a fluid within a neutralizing additive where the fluid may be or include a quench medium, an effluent stream, a dilution condensate, a dilution steam, and combinations thereof. However, it will be evident that various modifications and changes can be made thereto without departing from the broader spirit or scope of the invention as set forth in the appended claims. Accordingly, the specification is to be regarded in an illustrative rather than a restrictive sense. For example, specific neutralizable components, strong bases, amines, quench mediums, effluent streams, dilution condensates, and dilution steams falling within the claimed parameters, but not specifically identified or tried in a particular composition or method, are expected to be within the scope of this invention.

The present invention may suitably comprise, consist or consist essentially of the elements disclosed and may be practiced in the absence of an element not disclosed. For instance, the neutralizing additive composition for use in a fluid, such as but not limited to a quench medium, an effluent stream, a dilution condensate, a dilution steam, and combinations thereof may consist of or consist essentially at least one strong base and at least one amine; the strong base(s) may be or include metal hydroxides, metal carbonates, and combinations thereof.

The treated fluid composition may consist of or consist essentially of a fluid having neutralizable components and a neutralizing additive; the fluid may be or include a quench medium, an effluent stream, a dilution condensate, a dilution steam, and combinations thereof; the neutralizing additive may be present in the treated fluid composition in an amount effective to neutralize at least a portion of the neutralizable components; the neutralizing additive may have or include at least one strong base and at least one amine; the strong base may be or include metal hydroxides, metal carbonates, and combinations thereof; and more of the neutralizable components may be neutralized within the treated fluid composition as compared to an otherwise identical fluid composition absent the neutralizing additive composition.

The method may consist of or consist essentially of contacting a fluid having neutralizable components with a neutralizing additive composition in an effective amount to neutralize at least a portion of the neutralizable components, neutralizing at least a portion of the neutralizable components within the fluid; the fluid may be or include a quench medium, an effluent stream, a dilution steam condensate, a dilution steam, and combinations thereof; the neutralizing additive may include at least one strong base and at least one amine; the strong base(s) may be or include metal hydroxides, metal carbonates, and combinations thereof.

The words “comprising” and “comprises” as used throughout the claims, are to be interpreted to mean “including but not limited to” and “includes but not limited to”, respectively.

What is claimed is:

1. A neutralizing additive composition for use in a fluid selected from the group consisting of a quench medium, an effluent stream flowing from the quench water tower, the effluent from a process water stripper, a dilution condensate, a dilution steam, and combinations thereof; wherein the neutralizing additive comprises:
at least one strong base selected from the group consisting of metal hydroxides, metal carbonates, and combinations thereof; and
at least one amine;
quench medium (within the quench water tower), the effluent stream flowing from the quench water tower, the effluent from the process water stripper, the effluent from the dilution steam drum, the effluent from the dilution steam, and combinations thereof

2. The neutralizing additive composition of claim 1, wherein the at least one amine is selected from the group consisting of triethylenetetramine (TETA), tetraethylenepentamine (TEPA), diethanolamine (DEA), ethanolamine (MEA), methoxypropylamine (MOPA), morpholine, cyclohexylamine, and combinations thereof.

3. The neutralizing additive composition of claim 1, wherein the ratio of the strong base to the at least one amine ranges from about 1:60 to about 60:1.

4. The neutralizing additive composition of claim 1, wherein the metal hydroxides are selected from the group consisting of sodium hydroxide, calcium hydroxide, potassium hydroxide, lithium hydroxide, magnesium hydroxide, and combinations thereof; and wherein the metal carbonates are selected from the group consisting of sodium carbonate, calcium carbonate, potassium carbonate, lithium carbonate, magnesium carbonate, and combinations thereof.

5. The neutralizing additive composition of claim 1, wherein the amount of the at least one amine ranges from about 1 wt % to about 80 wt % based on the total amount of the neutralizing additive.

6. The neutralizing additive composition of claim 1, wherein the amount of the at least one strong base ranges from about 1 wt % to about 60 wt % based on the total amount of the neutralizing additive.

7. A treated fluid composition comprising:
a fluid comprising neutralizable components; wherein the fluid is selected from the group consisting of an effluent stream flowing from the quench water tower, the effluent from a process water stripper, a dilution condensate, a dilution steam, and combinations thereof; and
a neutralizing additive composition in an amount effective to neutralize at least a portion of the neutralizable components, the neutralizing additive composition comprising:
at least one strong base selected from the group consisting of metal hydroxides, metal carbonates, and combinations thereof; and
at least one amine; and
wherein more of the neutralizable components are neutralized as compared to an otherwise identical fluid composition absent the neutralizing additive composition.

8. The treated fluid composition of claim 7, wherein the at least one amine is selected from the group consisting of triethylenetetramine (TETA), tetraethylenepentamine (TEPA), diethanolamine (DEA), ethanolamine (MEA), methoxypropylamine (MOPA), morpholine, cyclohexylamine, and combinations thereof.

9. The treated fluid composition of claim 7, wherein the ratio of the strong base to the at least one amine ranges from about 1:60 to about 60:1.

10. The treated fluid composition of claim 7, wherein the metal hydroxides are selected from the group consisting of sodium hydroxide, calcium hydroxide, potassium hydroxide, lithium hydroxide, magnesium hydroxide, and combinations thereof; and wherein the metal carbonates are selected from the group consisting of sodium carbonate, calcium carbonate, potassium carbonate, lithium carbonate, magnesium carbonate, and combinations thereof.

11. The treated fluid composition of claim 7, wherein the neutralizing additive composition comprises the at least one amine in an amount ranging from about 1 wt % to about 80 wt % based on the total amount of the neutralizing additive.

12. The treated fluid medium composition of claim 7, wherein the neutralizing additive composition comprises the at least one strong base in an amount ranging from 1 wt % to about 60 wt % based on the total amount of the neutralizing additive.

13. A method comprising:
contacting a fluid having neutralizable components with a neutralizing additive composition in an effective amount to neutralize at least a portion of the neutralizable components; wherein the fluid is selected from the group consisting of an effluent stream flowing from the quench water tower, the effluent from a process water stripper, a dilution steam condensate, a dilution steam, and combinations thereof; wherein the neutralizing additive comprises at least one strong base and at least one amine; wherein the at least one strong base is selected from the group consisting of metal hydroxides, metal carbonates, and combinations thereof; and
neutralizing at least a portion of the neutralizable components within the fluid.

14. The method of claim 13, wherein the at least one amine is selected from the group consisting of triethylenetetramine (TETA), tetraethylenepentamine (TEPA), diethanolamine (DEA), ethanolamine (MEA), methoxypropylamine (MOPA), morpholine, cyclohexylamine, and combinations thereof.

15. The method of claim 13, wherein the ratio of the strong base to the at least one amine in the neutralizing additive composition ranges from about 1:60 to about 60:1.

16. The method of claim 13, wherein the metal hydroxides are selected from the group consisting of sodium hydroxide, calcium hydroxide, potassium hydroxide, lithium hydroxide, magnesium hydroxide, and combinations thereof; and wherein the metal carbonates are selected from the group consisting of sodium carbonate, calcium carbonate, potassium carbonate, lithium carbonate, magnesium carbonate, and combinations thereof.

17. The method of claim 13, wherein the neutralizing additive composition comprises the at least one amine in an amount ranging from about 1 wt % to about 80 wt % based on the total amount of the neutralizing additive.

18. The method of claim 13, wherein the neutralizing additive composition comprises the at least one strong base in an amount ranging from about 1 wt % to about 60 wt % based on the total amount of the neutralizing additive.
